(19) United States
(12) Patent Application Publication
(10) Pub. No.: US 2003/0104425 A1
(43) Pub. Date: Jun. 5, 2003

(54) Dna matrices and the use thereof for examining individuals of a population

(76) Inventor: Gottfried Brem, Hilgertshausen (DE)

Correspondence Address: BELL, BOYD & LLOYD, LLC, PO BOX 1135, CHICAGO, IL, 60690-1135, US

(21) Appl. No.: 10/169,294

(22) PCT Filed: Jan. 1, 2001
(86) PCT No.: PCT/EP01/00001

(30) Foreign Application Priority Data
Jan. 1, 2000 (DE) 100 00 001.0

(51) Int. Cl. C12Q 1/68; C12M 1/34

(52) U.S. Cl. 435/6; 435/287.2

(57) ABSTRACT

The present invention relates to the production of genotype arrays of a population on DNA matrices. The invention particularly relates to the use of such DNA matrices for determining features, certain genes, alleles, mutations, expression patterns, etc. in the individuals of each examined population.

The present invention relates to the production of genotype arrays of a population on DNA-matrices. In particular, the invention relates to the use of such DNA-matrices for determining features, particular genes, alleles, mutations, expression patterns, etc., of the individuals of each examined population.

Because of the successful development of molecular biology and of related techniques, it became possible to examine individuals for the presence of particular mutations in their genome. Thereby, the so-called DNA-chip technology gained acceptance, which technology is providing help to examine a plurality of mutations of an individual at once.

For said purpose, a chip which may be partitioned into hundreds of thousands of grid segments is provided with a particular number of cDNA fragments, synthetic oligonucleotides or other probes which are representing particular known mutations, and then a DNA sample collected from the individual to be examined will be examined on the chip.

In order to be able to perform this method in view of the costs, a plurality of identically constructed chips needs to be produced, i.e. chips which each are appropriate for the detection of the same known mutations.

However, this includes the disadvantage that a mutation newly discovered after the production of the chips, which mutation additionally shall be examined, i.e. the integration of a new gene into the chip, requires the production of new chips which additionally comprise this specificity, and allow the determination of this feature. The chips which are possibly still existing and do not have this new specificity are therefore usable only in a limited way.

In view of animal breeding, this means that if a new variant/marker is appearing, which will be from now on also interesting for all animals, e.g. if a new QTL (quantitative trait locus) has been discovered or an additional SNP (single nucleotide polymorphisms) became suddenly of interest for the breeding or other purposes, then for all animals previously genotyped with the help of a chip a subsequent typing work will have to be done.

In general, the analysis of known mutations, such as e.g. of the previously mentioned SNPs, requires the performing of a number of individual analyses or the use of a specific chip, by which chip these particular changes can be determined for each single individual. This involves, that millions of analyses or chips are required for examining a population for a particular feature and mutation, respectively.

If the examination will be performed by the help of probe chips by the means of the hybridization technique, then additionally the problem exists, that different probes hybridize at different temperatures with the corresponding counterpart on the matrix. This means that an analysis using the phenomenon of the melting temperature differences of sequences differing in one or several nucleotides when compared with the homologous sequences is not easily possible with the chip technology.

Moreover, another problem of the chip technology of today's date is that the genomic DNA samples of millions of not typed individuals have to be kept in order to be able to perform the analyses at a later point of time or to perform additional and more extended analyses, respectively. This is cost-intensive and requires a large storage space offer. Furthermore, also logistic problems will then be arising, if for a subsequent typing e.g. among the entire amount of samples single individuals will have to be searched for.

A problem of the present invention therefore consists in solving the above-mentioned problems.

According to the present invention, this problem will be solved by a method, wherein in a first step the genomic DNA of an individual of a population will be transferred on at least one DNA-matrix and this matrix will then be examined with a probe sensing the feature/mutation, in order to detect, whether an individual of the population has a particular feature and mutation, respectively.

In the figures,

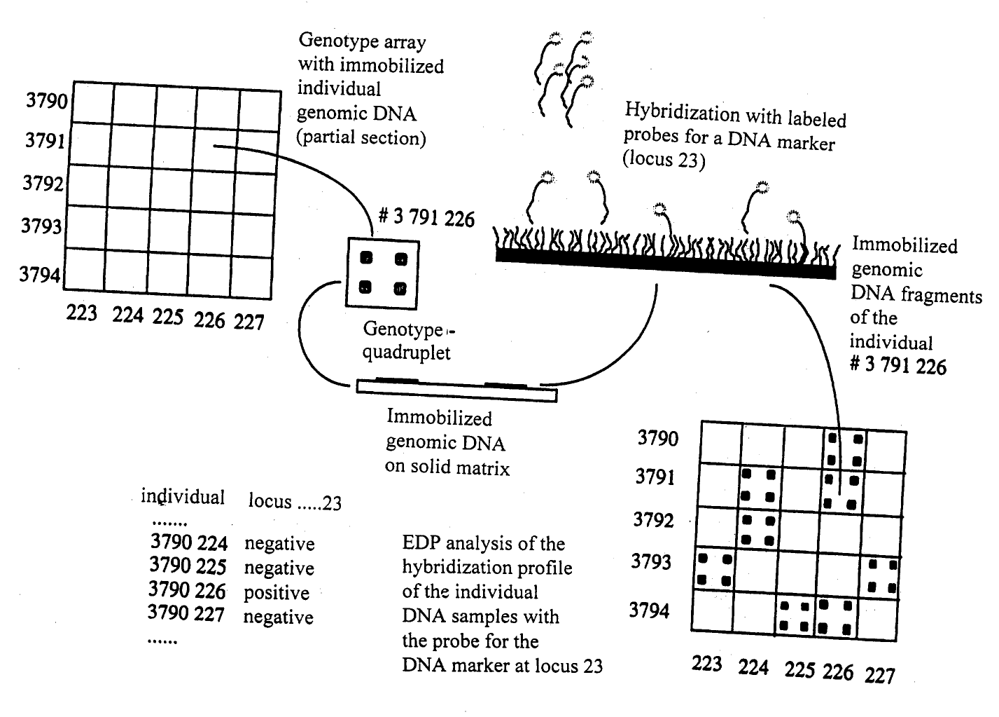

FIG. 1 shows schematically a matrix and the performance of the method.

A pre-condition for using the present invention is that DNA-containing samples of a particular parent population of individuals of a population, such as of particular parts or of all members of a section of the population, a stratum of society or a country, of animals, such as e.g. economically useful animals, such as e.g. cattle, pigs, etc. will be collected, and will be applied on the matrix and fixed there, respectively. The term population is therefore not only comprising a parent population of individuals, but also simply mating communities.

With respect to humans, the collection of DNA-containing samples may be done in a conventional way, e.g. by collecting samples of blood, saliva, mucosa, or skin, etc., the samples may already be conserved upon collection. Containers which are appropriate for the collection and conservation of DNA-containing samples are e.g. described in the DE 199 57 861.3. With respect to animals, the samples can be collected in the same way and manner or by the means of the sample collection system described in the WO 99/61882.

From the collected samples the DNA will be isolated and prepared according to conventional techniques (see e.g. Maniatis, 1992, Cold Spring Harbor, A Laboratory Manual) and with commercially available kits (such as e.g. Nucleo Spin Multi #740629,24,123813 from Mackery-Nagel), respectively. The collected DNA samples will then be prepared in such a way that they can be fixed with pipetting robots (e.g. the BioChipArrayer from Packard) on predetermined coordinates of the one or more matrices (see FIG. 1), whereby it is ensured that a particular coordinate on the DNA-matrix will be assigned to a particular individual.

As a matrix, the materials known in the state of the art are appropriate, such as e.g. glass, silicon, nylon, cellulose etc., with respect to the size of the matrix no limitations are being given. A matrix may have the size of the known DNA chips, but may also be larger. The size of the matrix to be used will substantially be depending on the number of populations to be examined, as well as on the capabilities of the available automated devices.

The application of the genomic DNA of the individuals (which are corresponding in their totality to the genotype of the individual) on the matrix may also be done in duplicate and quadruplet, respectively, in order to ameliorate the analyzability and to increase the accuracy, respectively, as a later statement on basis of an examination will only then be considered as correct, when all two and all four spots applied of the same genotype, respectively, will lead to the same result. However, these control analyses may be also achieved by real repetitions, by examining two or more identical chips with a probe, and by comparing the results.

As illustrated in FIG. 1, the matrix is partitioned in such a way into grid segments that each individual DNA sample which will be fixed on the matrix is assigned to a section and thereby can be identified. Thus, a collection of DNA samples (genotype array) with a very large number of genotypes on a single carrier will result. By using several carriers also populations comprising millions of individuals may be arranged on several few matrix units.

Newly added individuals of the population will be newly registered on additional units of the matrix, should the occasion arise, in particular periods (e.g. monthly or annual), so that the parent population will essentially always stay completed. This collection of matrix units (genotype arrays, genome chips) represents therefore the genotypes of an entire population. From each genotype matrix a large number of copies will be made, which may then be used for the analyses.

The population registered in such way may then be examined for particular features, such as e.g. the presence of particular alleles, mutations, the predisposition to develop particular diseases and the resistance against diseases, etc., respectively.

For this purpose, the at least one chip, i.e. the complete set of matrices, which is carrying the totality of the genotypes of this particular population, will be examined with a specific probe, which allows with respect to the feature a specific statement.

As a probe e.g. a segment of a (mutated) gene well known to cause a particular predisposition for a particular disease, such as e.g. MHS for pigs, BLAD for cattle, etc. may be used.

For a visual measurement, when the occasion arises, these probes can be connected to conventional materials, such as radioactive isotopes, colored and fluorescent substances.

However, apart from hybridization with the probe, the set of genotype matrices can also be used for directly performing on the carrier and analyzing a PCR, a TMA (Transcription Mediated Amplification), a bDNA reaction (branched DNA) or another method for specific DNA detections.

Thus, via e.g. the “solid phase PCR” all individuals of a population may be examined in a single set-up with respect to a feature of interest, which provides enormous advantages with respect to the costs, as the expenses for the PCR set-up for the individuals of the entire population will then not be essentially higher than for a PCR reaction for one individual, since millions of individuals will be analyzed in a single PCR reaction with respect to a variant.

In general, the analysis of the genotype matrices after hybridization or PCR reaction is performed by an automated device, as the error rate would be too high, which would occur when manually assigning the miniaturized, complex result pattern on the matrices to the individuals. The hybridization or PCR pattern will therefore be sensed by a scanner and analyzed by an image analysis system.

For this, hard- and software systems are appropriate, which have been developed for analyzing conventional DNA chips and are already used therefore. By the position on the matrix, it is generally fixed which individual is present there and the result of the hybridization or PCR reaction on this position will be associated with the identity of the individual and shown in an analysis protocol or an analysis file.

For further controlling and assuring the analysis, single individuals which are also present on the matrices, may be determined in a separately set-up, individual, conventional singular PCR reaction, or another detection method with separately stored DNA, and may then be used as positive and negative controls for the matrix set-up.

The results of the matrix analyses which can be stored in files, when the occasion arises, may then be provided to authorized persons or institutions for retrieving.

The DNA chips of the present invention may also be used for the determination of the lack of side effects and the responsiveness of medicaments, respectively.

Effective medicaments may sometimes act in the body as poisons. The recommended dosage may be effective in particular individuals, may not have any positive effect at all in others, and, in turn, be toxic or even lethal in others. Side effects of medicaments are well known to be among the ten most frequent causes of death of humans.

The aim of modern pharmacogenetics is therefore to match the administration of medicaments to the individual genotype. Fine genetic differences such as e.g. particular SNPs, which may influence these effects must therefore be detected and analyzed.

For each medicament which is approved or to be newly launched on the market, it may now be examined with the help of the present invention already before a launching on the market—as far as the corresponding genetic components are known and the genotype arrays are present—with which certainty it will be effective, which and how many individuals, respectively, may not be cured, and to what extent incompatibilities will arise.

In a special case, it may therefore be determined before the treatment of an individual by retrieving the above described EDP data file, wherein the results of the SNP analysis for the concerned medicament are stored, whether the chosen medicament is appropriate for providing help to this patient and which amount of dosage seems to be appropriate or whether another medicament is to be preferred.

This procedure is in particular in case of populations of economically useful animals of great interest, as for animals until now no possibility is present to perform such examinations. According to the present invention, the pharmacogenetic analysis may be performed at low costs, so that it will also be of interest for animals.

The above described procedure which represents, when compared to known systems, a complete change of paradigm, has in summary the following advantages:

1) The population matrices may be stored easily, without any problems, without taking up a lot of space, at low costs and for a long time, i.e. nearly unlimited. This is very advantageous, when compared to conventional methods, wherein the storage of millions of samples is causing enormous logistic problems and relatively high costs.

2) These matrices may then be examined and analyzed, respectively, in toto each with a single probe, e.g. by hybridization or another technique, such as e.g. PCR, LCR, bDNA, TMA or similar with respect to a particular feature, a variant or mutation, etc.

3) Thus, only relatively few individual analyses will be required, depending nearly exclusively from the number of different individual typing processes and not from the number of probands. Consequently, even millions of individuals may be easily typed.

4) The melting temperature differences during the hybridization with not unambigously corresponding sequences may be excellently used for the detection of base substitutions. As per matrix only one probe is used, the conditions of hybridization, such as e.g. the temperature for the respective probe may be optimally chosen. The skilled person will choose the appropriate hybridization temperature on basis of his general knowledge in the art, taking the length of the probe, as well as the G/C-content thereof into consideration.

5) Subsequent typings with newly discovered markers, mutations, etc., may be performed in a single set-up for the entire population registered at that moment at any time and at very low costs, without a renewed recourse to the individual samples of the individuals being required. An analysis of the chips/matrices prepared on stock allows that this procedure is completely unproblematic. When using the present invention, after the discovery/publication of a novel marker and the decision to use it, during e.g. a week the entire population may be typed and the results for millions of single individuals will be available for interested/authorized persons.

The following examples are illustrating the invention and are not intended to be limiting the invention in any way.

### EXAMPLE 1

Origin Ascertainment and Marker Genotyping of Economically Useful Animals

The European Union (EU) labeling regulation lays down that economically useful animals such as e.g. cattle have to be marked with double ear tags in the European Union. These ear tags have to be inserted during the first week of life. When using this labeling by ear tags for collecting a tissue sample of the animals, what may be easily achieved by the means of the system described in the WO 99/61882, a genotype collection of all cattle of the European Union may be built up easily and at low costs. This means that after a particular time all 80 million cattle of the European Union will be collected on these genotype matrices, newly added calves each being collected on new matrices and assigned to the pool.

The set of matrices of the economically useful animal species may now be examined with 50 different SNP markers (for each marker a set). Thereby, a genetic fingerprint may be registered for all 80 million animals, which fingerprint allows to find unambiguously among the milliards of genotypes a single animal and its identity, respectively, and also to ascertain the descent when the occasion arises.

The same applies for pigs, sheep, goats, camelides, horses, rabbits, birds, etc.

Analyses with particular markers may be used to determine for all cattle of the European Union, which genetic constellation they have e.g. in view of various milk protein genes or to find out which animals are carriers of positive QTLs, whether they have particular genetic mutations (e.g. BLAD), so that they are deemed to be appropriate models for human diseases, e.g. for particular hemoglobin variants, whether they have specific genetic incompatibilities, how they react upon a treatment with medicaments, which medicament is optimal for a particular disease for this genotype, which dosage is helpful, which animals have dispositions for or resistances against particular disease causing agents or disease influences, etc.

Apart from the previously described possibilities, for pigs e.g. a typing of all animals of a breed or a population may be carried out easily, quickly and at low costs with respect to the genotype for MHS, estrogen receptor variants, intramuscular fat gene loci, particular variants providing advantages for xenotransplantations, etc.

Moreover, via the proposed system a simple, reliable and quick ascertainment of the descent, of the origin and of the identity for all animals may be carried out and updated.

### EXAMPLE 2

Detection of Mixing-Ups During the Marketing of Meat

More and more food groups and distributors of meat and meat products are meanwhile guarantying for the indicated origin of their products, e.g. that the animals are animals which are born and raised inland, or that the products are made thereof, or that the products are products from particular biologic particularly estimable production units. By this unambiguous and checkable ascertainment of origin and identity (see WO 99/61882) such mixing-ups or possibly occurring deceitful exchanges may be detected.

In the course of the slaughtering and in particular during and after the cutting-up and marketing the meat has passed through many hands, whereby it may often not be determined any more who is responsible for the mixing-up. This may be solved by taking reserve samples at each change from one market participant to the next one. In case of problems, it may be then determined by a later analysis when the identity of the sample got lost.

### EXAMPLE 3

Import of Carcasses and Meat from Foreign Countries

Because of the problems with BSE, after the revocation of the ban on imports which was decreed by the Commission in Brussels, currently some countries are fighting against beef from foreign countries which could be entering from now on the domestic market and could be offered there without being recognized, so that the security and health, respectively, of the domestic consumers could be at risk.

In spite of a possible marking of the products originating from foreign countries, the consumers still have reservations and feelings of insecurity as the markings of meat from foreign countries could be removed and the meat could be marketed in spite of this being forbidden as a domestic product.

For the uncovering of such deceitful/falsifying labeling, meat samples from all imported carcasses could be collected, DNA could be isolated thereof, and used for the preparation of genotype matrices. This is also possible for frozen products.

In case of a suspicion, DNA of the suspected sample will be compared with the genotypes conserved on matrices, whereby it may be determined easily and at low costs, whether the suspected sample is identical with a reserve sample of imported meat or not.

### EXAMPLE 4

Examination with Respect to an Addition of Transgenic Products

In case of transgenic cereals imported from overseas to Europe, it often occurs, that non-transgenic cereals are mixed with a small amount of transgenic cereals.

For the examination of such cases, from all arriving loads of cereals about ten thousand grains will be taken and conserved individually or pooled after DNA-preparation on a matrix. In case of a suspicion, samples of cereals which will be collected during the further marketing and processing of the cereals, may be assigned via a comparison with the genotypes on the matrices of unambiguously determined deliveries and concrete transgenic changes.

Similar examinations may also be desired for transgenic animals and the products thereof.

### EXAMPLE 5

DNA Collections

Meanwhile the legal basis was provided in a number of countries, which legal basis allows to keep DNA samples of a particular group of persons. These DNA samples may be applied on genotype matrices and then be referred to for examinations in case of a suspicion, so in case of similar violations of the law.

### EXAMPLE 6

Data Collection of Particular Individuals of a Population

In particular regions or countries, DNA samples of all inhabitants could be collected with their consent and applied on genotype matrices. Such a consent has already been achieved in Iceland, so that samples of all inhabitants may be collected there, stored and analyzed after anonymization. In case of an appropriate legal situation according to data protection law, also in other countries a basis could be created which allows that DNA samples of a human subpopulation or population group are collected on a voluntary basis and will then be used for medical applications (e.g. for the identification of the appropriate medicament, the appropriate dosage, the detection of genetic incompatibilities or problems, etc.). The present invention consequently shows how these samples, if they are collected then, may be stored and analyzed optimally and at low costs.

1. A method for examining individuals of a population using DNA-matrices, wherein
the genomic DNA of essentially all individuals of a population being fixed on at least one matrix, such that a particular identifiable segment on the matrix is assigned to each individual, and
the matrix being examined with a probe of interest.

2. The method of claim 1, wherein the DNA samples being applied in a duplicate and a quadruplet, respectively, on the DNA-matrix.

3. The method of claim 1 or claim 2, wherein the examination with a probe is performed by the means of hybridization, PCR, TMA, or a bDNA reaction.

4. The method of one of the preceding claims, wherein the DNA-matrix has been stored prior to use.

5. A DNA-matrix having a carrier, on which carrier DNA may be bound, characterized in that it includes genomic DNA of individuals of a particular population in predetermined, identifiable segments.

6. The DNA-matrix of claim 4, characterized in that the population is economically useful animals.

7. Use of a DNA-matrix of one of the claims 5 or 6 for examining an individual with respect to a particular feature.

8. The use of claim 7, wherein the feature being a predisposition for and a resistance against a particular disease, respectively, or the lack of side effects of and the responsiveness to a particular medicament, respectively.

9. The use of claim 7, wherein the feature is based on a mutation in a gene or the presence of a particular allele.

10. The use of a DNA-matrix of one of the claims 6 or 7 for ascertaining the origin and for marker genotyping of economically useful animals, for detecting mixing-ups in the marketing of meat, for examining products for the addition of transgenic starting materials, for collecting DNA data of particular individuals of the population and for collecting population data.
